(19) United States
(12) Patent Application Publication
(10) Pub. No.: US 2014/0234447 A1
(43) Pub. Date: Aug. 21, 2014

(54) COMPOSITION TO BE APPLIED TO THE SKIN, AND USE THEREOF

(71) Applicant: ALNAPHARM GMBH & CO. KG, Hamburg (DE)

(72) Inventor: Ali Nahavandi, Hamburg (DE)

(21) Appl. No.: 14/347,788

(22) PCT Filed: Sep. 10, 2012
(86) PCT No.: PCT/EP2012/003802

§ 371 Date: Mar. 27, 2014

(30) Foreign Application Priority Data
Sep. 30, 2011 (EP) 11007992.8

(51) Int. Cl. A61K 36/889; A61K 36/736; A61K 36/53; A61K 36/185

(52) U.S. Cl. CPC A61K 36/889; A61K 36/185; A61K 36/736; A61K 36/53
USPC 424/735; 424/727; 424/776; 424/745

(57) ABSTRACT

The present invention concerns a composition to be applied to the skin, which comprises a dermatologically compatible vehicle, coconut oil, hazelnut oil and/or avellana oil, and stinging nettle oil, as well as use thereof for the treatment of skin diseases.

The present invention relates to a composition to be applied to the skin, as well as the use thereof in the treatment of skin diseases.

Psoriasis and neurodermatitis (atopic eczema) are very common, chronic, non-contagious, inflammatory skin diseases.

Thus for example around 2 to 3% of the population suffer from the most frequently occurring psoriasis vulgaris, with psoriasis pustolosa being likewise well-known. Women and men are affected equally. Psoriasis is a chronic skin disease, which causes lifelong symptoms. Around one in five psoriasis patients suffer additional arthritic symptoms (psoriasis arthritis) and other chronic inflammatory diseases. Because of the interaction of various clinical pictures and symptoms, life expectancy can be reduced. For both these diseases, the causes and triggers have not yet been conclusively established. Various theories are discussed in the literature. Genetic factors, immunological changes and/or environmental influences are said to play a significant role.

External influences can be very diverse. Mechanical, infectious, medication-related, psychological and chronic inflammations are seen as trigger factors.

For psoriasis, one possible explanation for the disease is that the body's own defence system is disrupted because of immune responses. Here, the production of the T-cells that are responsible for the body's defence responses proceeds unchecked, whilst T-cell production is however a decisive factor in the regulation of the defence system of the skin. In psoriasis, an uncontrolled antibody defence response develops, and not only are exogenous agents attacked, but also those of the body itself. There is a malfunction in the reproduction of skin cells. Affected skin areas can show severe irritation, reddening, silvery-scaly deposits, and are thickened in places. Some patients have cracked skin and open wounds.

In healthy skin, the upper layer of the skin (the epidermis) is renewed at regular intervals. Here, new skin cells are formed, which then age and become hardened. The hardened skin cells (keratinocytes) are cast off by the body. In the case of a healthy body, this process proceeds almost unnoticed and unseen. In healthy skin, the keratinocytes form a natural protective shield against external environmental influences. The repair mechanism for healthy skin acts via targeted direction of the keratinocyte formation and activation of the T-cells. By contrast, in the case of psoriasis cell growth is disrupted. The formation of skin cells is heavily accelerated, and a disproportionately large number of cells is formed. The increased keratinocyte formation is activated without any outside action, and continues in an uncontrolled manner. A shiny, silvery-white scaly layer forms on the skin. The lower levels of the skin have enhanced blood circulation on account of the uncontrolled cell growth, and thus appear severely reddened.

The pathological skin changes (plaques) are frequently distributed individually, in an insular manner. The skin areas most frequently affected are those which are stretched and are subjected to continual mechanical stress. The skin are as become thickened and form scales. Through the scale formation, the skin becomes hardened and has a tendency towards dryness and wounds.

It is not possible to cure psoriasis. There are many different treatment approaches aimed at soothing the symptoms. The treatments depend on the severity, location and spread of the lesions. Local and systemic treatments are used, and these can be in the form of preparations or they can be physical. Fundamentally, for initial symptoms and for general care, moisturising skin care products in the form of lotions, creams, oils and ointments are used. If the condition is more advanced, external (topical), internal (systemic) treatments and light are used as forms of treatment. What all these measures have in common is the intention to suppress the scale formation and development of inflammation. The aim is to restore a normal balance of immune response.

However, particularly in the case of systemic therapeutic treatment, it is possible for this to have far-reaching effects on the body, and this requires extremely thorough observation and monitoring, since serious complications and side effects can occur.

One task of the present invention is to provide a composition that is to be applied to the skin, which overcomes the disadvantages of the prior art, and which in particular enables an extremely gentle way of restoring the natural protective function of the skin. The intention here is, in particular, to regenerate the natural barrier function of the skin, and to maintain the processes present in healthy skin. Here, the composition according to the invention should preferably be capable of being used to support the therapeutic treatment of psoriasis and neurodermatitis.

This problem is solved by a composition to be applied to the skin, which comprises a dermatologically compatible vehicle, coconut oil, hazelnut oil and/or avellana oil, and stinging nettle oil.

It is preferably envisaged here that the weight ratio of dermatologically compatible vehicle:coconut oil:hazelnut oil and/or avellana oil:stinging nettle oil lies within a range of 1-50:1-30:1-50:1-10.

Furthermore, it is preferably envisaged that the composition comprises almond oil.

It is preferably envisaged here that the weight ratio of dermatologically compatible vehicle:coconut oil:hazelnut oil and/or avellana oil:stinging nettle oil:almond oil lies within a range of 1-50:1-30:1-50:1-10:1-20.

Furthermore, it is preferably envisaged that the composition comprises TRF extract (tocotrienol-rich fraction).

It is particularly preferred here that the weight ratio of vehicle:coconut oil:hazelnut oil and/or avellana oil:stinging nettle oil:almond oil:TRF extract lies within a range of 1-50:1-30:1-50:1-10:0-20:1-20.

It can also be envisaged that the composition comprises oil of bitter almonds.

It is preferably envisaged here that the weight ratio of vehicle:coconut oil:hazelnut oil and/or avellana oil:stinging nettle oil:almond oil:TRF extract:oil of bitter almonds lies within a range of 1-50:1-30:1-50:1-10:0-20:0-20:1-10.

It is furthermore proposed that the composition comprises natural aromatics, preferably lavender aroma.

For preference, it is envisaged here that the weight ratio of vehicle:coconut oil:hazelnut oil and/or avellana oil:stinging nettle oil:almond oil:TRF extract:oil of bitter almonds:natural aromatics lies within a range of 1-50:1-30:1-50:1-10:0-20:0-20:0-10:0.1-1.

For particular preference, it is envisaged that the composition comprises:

| | |
|---|---|
| Dermatologically | 1-50% by weight, preferably 30-50% by |
| compatible vehicle | weight, |
| | even more preferably 40.00% by weight, |
| Coconut oil | 1-30% by weight, preferably 10-30% by |
| | weight, |
| | even more preferably 15-25% by weight, |
| | even more preferably 17.50% by weight, |
| Hazelnut oil and/o | 1-50% by weight, preferably 10-40% by |
| ravellana oil | weight, |
| | even more preferably 15-35% by weight, |
| | even more preferably 20-30% by weight, |
| | even more preferably 25.00% by weight, |
| Stinging nettle oil | 1-10% by weight, preferably 1-7% by |
| | weight, |
| | even more preferably 3.00% by weight, |
| Almond oil | 0-20% by weight, preferably 5-15% by |
| | weight, |
| | even more preferably 10.00% by weight, |
| TRF extract (tocotrienol- | 0-20% by weight, preferably 1-10% by |
| rich fraction) | weight, |
| | even more preferably 2-7% by weight, |
| | even more preferably 3.00% by weight, |
| Oil of bitter almonds | 0-10% by weight, preferably 0.5-3% by |
| | weight, |
| | even more preferably 1.00% by weight, |
| Aromatics | 0-1% by weight, preferably 0.3-0.7% by |
| | weight, |
| | even more preferably 0.50% by weight, |

wherein all the percentages by weight relate to the total
quantity of the composition.

If the composition according to the invention contains dermatologically compatible vehicles, coconut oil, hazelnut oil and/or avellana oil, stinging nettle oil and oil of bitter almonds, the weight ratios preferably lie within a range of 1-50:1-30:1-50:1-10:1-10.

It can furthermore be envisaged that it is presented in the form of an ointment, cream, lotion, tincture, oil or gel.

In principle, any dermatologically compatible vehicle that is suitable for the production of ointments, creams, lotions, tinctures, oils or gel scan be used. Experts in the field know of corresponding dermatologically compatible vehicles.

Here, it can preferably be envisaged that the dermatologically compatible vehicles selected from the groups:
a. Hydrophobic ointments
   for example comprising: white Vaseline Ph. Eur., yellow Vaseline Ph. Eur, simple ophthalmic ointment DAC
b. Lipophilic gels
   for example comprising: hydrophobic base gel DAC
c. lipogels
   for example comprising: lard DAB, white almond oil ointment FH A.4, excipial almond oil ointment
d. water-absorbing ointments W/O absorption ointments
   for example comprising: wool wax alcohol ointment DAB (Ungt. Alcohol. Lanae), Eucerinum Abhydricum, Ungt. Sorbitansesquioleati, Ungt. Sorbitanmonostearinic, wool wax-free W/O-absorption ointment, Pionier KWH pharma, emulsifying hydrophobic base gel DAC, emulsifying eye ointment (NRF 15.20)
e. O/W absorption ointments
   for example comprising: hydrophilic ointment DAB, Unguentum Cordes
f. Lipophilic creams
   for example comprising: lanolin DAB, oily cream (Ungt. Alcoholum Lanae aquosum), Eucerin cum aqua, emollient ointment (Ungt. Molle) DAC, hydrophobic base cream DAC (NRF 11.104), hydrophobic tretinoin cream 0.025/0.05 or 0.1% (NRF 11.123), hydrophobic triclosan cream 2% (NRF 11.122), hydrophobic polidocanol cream 5% (NRF 11.119), hydrophobic polidocanol cream 5% with urea 5% (NRF 11.120), Cremor vaselini MB 59, Cremor sorbitansequioleati, Cremor sorbitanmonostearati,
g. W/O lotions
h. Quasi-W/0 creams
   for example comprising: cold cream (Ungt. Leniens) DAB, cold cream naturel RP
i. Hydrophilic creams
   for example comprising: non-ionic hydrophilic cream DAB, non-ionic hydrophilic cream SR DAC (NRF S.27), non-ionic aqueous liniment DAC (NRF 11.92)
j. Hydrophilic lotions
   for example comprising: hydrophilic base emulsion (NRF S.25)
k. Hydrophilic gels
   for example comprising: hydroxyethyl cellulose gel DAB

A second problem is solved by the use of the composition for the treatment of skin diseases, in particular psoriasis, neurodermatitis (atopic dermatitis), seborrhoeic dermatitis, urticaria, erythema and lichen planus, as well as for the treatment of wounds/skin burns and corns.

Surprisingly, it was found that the composition according to the invention soothes symptoms associated with skin diseases, such as in particular psoriasis and neurodermatitis. Moreover, the composition according to the invention accelerates the healing of wounds/skin burns as well as corns. In the opinion of the inventors, this takes place on account of physical effects. The composition is based on natural oils as well as a conventional vehicle for the manufacture of the composition, in order to make this suitable for topical application. In combination, the ingredients have a positive effect on the regeneration of natural skin functions. The soothing effect is rather achieved through moisturising and caring effects. When applied to the skin, the composition produces a protective film that protects the affected skin areas from external environmental influences and supports the body's own regeneration of skin functions. Through the formation of a protective film, the increased drying of the lesions is stopped, and the water content in the skin layers can be regenerated. In particular, the water content in the corneum (Stratum corneum) is a decisive factor for healthy skin. The epidermis of healthy skin has natural barrier functions which regulate the water equilibrium, and protect the skin from environmental influences and harmful substances. However, if the skin is affected by psoriasis or neurodermatitis, the natural barrier function is impaired. The composition according to the invention accelerates the restoration of the normal barrier function of the skin. The composition according to the invention protects the skin from harmful environmental influences and substances that trigger allergies. The lipid components contained in the composition according to the invention also produce a cooling effect, resulting in additional soothing.

The effects of the composition according to the invention mean that the skin can regenerate, the formation of the skin's natural barrier function is supported, and the natural protective barrier function of healthy skin is restored.

Further features and advantages of the composition according to the invention follow from the following detailed description of preferred embodiments.

Example production of a composition as a cream:

Into a dermatologically compatible vehicle, in this case for example Eucerin anhydricum, the components, the oils listed in the composition, are added one after another, whilst stirring, and these are worked into the vehicle. The quantitative ratios of the components result from the number of ingredients and the size of the batch. The quantitative proportions result from the desired batch size. The weight quantities are calculated from the percentages by weight in relation to the batch size.

Depending on the dermatological vehicle used and the number and quantity ratio of the oils used, the result is an oily or creamy structure of the composition.

Compositions used in percentages by weight, for testing efficacy:

| | | | | Stinging | | | Oil of | |
|---|---|---|---|---|---|---|---|---|
| | Eucerin | Coconut | Hazelnut | nettle | Almond | TRF | bitter | Lavender |
| No. | anhydricum | oil | oil | oil | oil | extract | almonds | oil |
| 1 | 40.0 | 17.5 | 25.0 | 3.0 | 10.0 | 3.0 | 1.0 | 0.5 |
| 2 | 10 | 13 | 50 | 1 | 1 | 20 | 5 | 0 |
| 3 | 20 | 30 | 40 | 5 | 5 | 0 | 0 | 0 |
| 4 | 30 | 6 | 20 | 8 | 15 | 10 | 10 | 1 |
| 5 | 40 | 10 | 10 | 10 | 20 | 5 | 5 | 0 |
| 6 | 40 | 27 | 30 | 3 | 0 | 0 | 0 | 0 |
| 7 | 50 | 5 | 25 | 2 | 15 | 0 | 2.5 | 0.5 |

The cream compositions listed in the table were used to test efficacy in 49 patients with psoriasis, 33 patients with neurodermatitis as well as 28 patients for the treatment of wounds/skin burns and corns. The creams with the example compositions (see above) were applied 1-3 times a day.

The effect of the compositions on the diseased skin was assessed in 49 patients with psoriasis, at intervals of 1, 5, 10, 20, 30, 45, 60, 75 and 90 days after the beginning of application.

The results of the effect observed are shown in the following table.

Efficacy in the case of psoriasis

| Compo- | | | | | | | | | |
|---|---|---|---|---|---|---|---|---|---|
| sition | 1 | 5 | 10 | 20 | 30 | 45 | 60 | 75 | 90 |
| no. | day | days | days | days | days | days | days | days | days |
| 1 | +++ | +++ | ++++ | ++++ | ++++ | ++++ | ++++ | ++++ | ++++ |
| 2 | + | ++ | +++ | ++++ | ++++ | ++++ | ++++ | ++++ | ++++ |
| 3 | + | +++ | +++ | +++ | +++ | +++ | ++++ | ++++ | ++++ |
| 4 | −/+ | + | + | ++ | ++ | +++ | ++++ | ++++ | ++++ |
| 5 | − | − | −/+ | + | ++ | ++ | +++ | ++++ | ++++ |
| 6 | ++ | +++ | ++++ | ++++ | ++++ | ++++ | ++++ | ++++ | ++++ |
| 7 | + | ++ | +++ | +++ | +++ | +++ | ++++ | ++++ | ++++ |

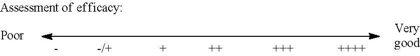

The effect of the compositions on the diseased skin was assessed, for 33 patients with neurodermatitis, at intervals of 5, 10, 14, 22, 30, 45, 60, 75 and 90 days after the beginning of application.

The results of the observed effect are shown in the following table.

Efficacy in the case of neurodermatitis

| Compo- | | | | | | | | | |
|---|---|---|---|---|---|---|---|---|---|
| sition | 5 | 10 | 14 | 20 | 30 | 45 | 60 | 75 | 90 |
| no. | days | days | days | days | days | days | days | days | days |
| 1 | + | ++ | ++ | +++ | +++ | +++ | ++++ | ++++ | ++++ |
| 2 | + | + | + | ++ | ++ | +++ | +++ | ++++ | ++++ |
| 3 | + | + | + | + | ++ | +++ | +++ | ++++ | ++++ |
| 4 | −/+ | −/+ | + | ++ | ++ | ++ | +++ | ++++ | ++++ |
| 5 | − | −/+ | −/+ | + | ++ | ++ | +++ | +++ | ++++ |
| 6 | + | + | ++ | +++ | +++ | +++ | +++ | ++++ | ++++ |
| 7 | −/+ | + | + | ++ | ++ | +++ | +++ | ++++ | ++++ |

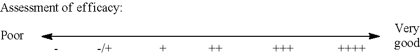

The effect of the compositions on the diseased skin was assessed in 28 patients with wounds/skin burns as well as corns, at intervals of 1, 2, 5, 7, 9, 10 and 14 days after the beginning of application.

The results of the effect observed are shown in the following table.

Wounds/skin burns/corns

| Composition | 1 | 2 | 5 | 7 | 9 | 10 | 14 |
|---|---|---|---|---|---|---|---|
| no. | day | days | days | days | days | days | days |
| 1 | ++ | +++ | +++ | ++++ | ++++ | ++++ | ++++ |
| 2 | ++ | ++ | ++ | +++ | +++ | ++++ | ++++ |
| 3 | ++ | +++ | +++ | +++ | +++ | ++++ | ++++ |
| 4 | + | + | + | ++ | ++ | +++ | +++ |
| 5 | −/+ | −/+ | −/+ | + | + | ++ | +++ |
| 6 | ++ | +++ | ++++ | ++++ | ++++ | ++++ | ++++ |
| 7 | + | + | ++ | ++ | +++ | +++ | ++++ |

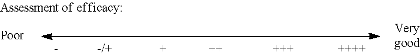

From the results for the compositions which are shown above, it can be seen that all the compositions listed bring about soothing in psoriasis, neurodermatitis and in the treatment of wounds/skin burns as well as corns. The composition that is preferably envisaged has the greatest, most universal and most comprehensive efficacy in all the envisaged areas of application.

Furthermore, comparison compositions were produced which comprised a dermatologically compatible vehicle (Eucerin anhydricum) and, on the one hand, respectively only hazelnut oil or coconut oil, and on the other hand a mixture of hazelnut oil/coconut oil, and their efficacy in the case of psoriasis patients was observed.

In addition, corresponding trials were carried out based on “vehicle+stinging nettle oil”, “vehicle+stinging nettle oil 3%+coconut oil” and “vehicle+stinging nettle oil 3%+hazelnut oil”, and the compositions that were produced were observed in respect of their efficacy in the case of psoriasis patients.

Finally, a composition according to the invention, of a dermatologically compatible vehicle, coconut oil, hazelnut oil and stinging nettle oil, was produced and likewise investigated.

Dermatologically Compatible Vehicle+Hazelnut Oil

A dermatologically compatible vehicle, Eucerin anhydricum, was mixed with hazelnut oil in the proportions shown in the table below, with the percentages being percentages by weight. The mode of action of the composition produced in this way in the treatment of psoriasis was tested. For this, 30 people with mild to moderate psoriasis were treated with this composition over a period of 30 days. In order to enable the treatment to be observed, different vehicle/oil proportions were applied to different areas of skin, and the effect was observed. The results are shown in the following table:

Trial of vehicle + hazelnut oil: Efficacy in the case of psoriasis

| Oil content in percent | 1 day | 5 days | 10 days | 20 days | 30 days |
|---|---|---|---|---|---|
| 10 | − | − | − | −/+ | + |
| 20 | − | − | −/+ | −/+ | + |
| 30 | − | −/+ | −/+ | −/+/ | + |
| 40 | − | −/+ | −/+ | + | + |
| 50 | − | −/+ | + | + | + |

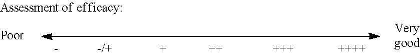

Dermatologically Compatible Vehicle+Coconut Oil

Analogously to the investigations of a composition of vehicle+hazelnut oil as described above, corresponding investigations were also carried out for a composition of just the vehicle and coconut oil. The results are shown in the following table:

Trial of vehicle + coconut oil: Efficacy in the case of psoriasis

| Oil content in percent | 1 day | 5 days | 10 days | 20 days | 30 days |
|---|---|---|---|---|---|
| 10 | − | − | − | −/+ | + |
| 20 | − | − | − | −/+ | + |
| 30 | − | − | −/+ | −/+ | + |
| 40 | − | − | −/+ | + | + |
| 50 | − | −/+ | −/+ | + | + |

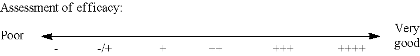

Vehicle+Hazelnut Oil+Coconut Oil

A composition of vehicle, hazelnut oil and coconut oil was produced, with the two oils being added to the vehicle in equal proportions by weight. The oil proportions given in the table below are calculated from the sum of the individual oil components.

The results are as follows:

Trial of vehicle + hazelnut oil + coconut oil:
Efficacy in the case of psoriasis

| Oil content in percent | 1 day | 5 days | 10 days | 20 days | 30 days |
|---|---|---|---|---|---|
| 10 | − | − | − | −/+ | + |
| 20 | − | − | + | + | + |
| 30 | −/+ | + | + | ++ | ++ |
| 40 | −/+ | + | + | ++ | +++ |
| 50 | −/+ | + | + | ++ | +++ |

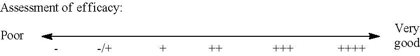

Of the compositions tested in this connection, the composition with an oil content of 40% (with the same proportions by weight of hazelnut oil and coconut oil) proved to be the most effective.

Trial of vehicle + stinging nettle oil: Efficacy in the case of psoriasis

| Oil content in percent | 1 day | 5 days | 10 days | 20 days | 30 days |
|---|---|---|---|---|---|
| 1 | − | − | − | −/+ | + |
| 2 | − | − | −/+ | + | + |
| 3 | − | −/+ | + | + | ++ |
| 5 | − | −/+ | + | + | ++ |
| 10 | − | −/+ | −/+ | + | ++ |

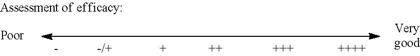

Trial of vehicle + stinging nettle oil 3% + coconut oil:
Efficacy in the case of psoriasis

| Coconut oil content | | | | | |
|---|---|---|---|---|---|
| in percent | 1 day | 5 days | 10 days | 20 days | 30 days |
| 10 | − | − | −/+ | + | ++ |
| 20 | − | −/+ | + | + | ++ |
| 30 | − | + | + | ++ | ++ |
| 40 | − | + | + | ++ | ++ |
| 50 | −/+ | + | + | ++ | ++ |

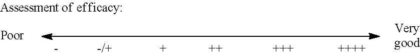

Trial of vehicle + stinging nettle oil 3% + hazelnut oil:
Efficacy in the case of psoriasis

| Hazelnut oil content | | | | | |
|---|---|---|---|---|---|
| in percent | 1 day | 5 days | 10 days | 20 days | 30 days |
| 10 | − | −/+ | −/+ | + | + |
| 20 | − | −/+ | + | + | + |
| 30 | −/+ | + | + | ++ | ++ |
| 40 | −/+ | + | + | ++ | +++ |
| 50 | −/+ | + | + | ++ | +++ |

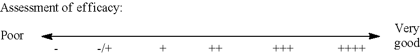

Vehicle+Hazelnut Oil+Coconut Oil+Stinging Nettle Oil (According to the Invention)

Starting out from the composition, described above, with a 40% oil content (equal proportions of hazelnut oil and coconut oil), stinging nettle oil was added to this composition, so that a composition according to the invention was produced.

The following table shows the results for the treatment of psoriasis with the composition according to the invention:

Trial of vehicle + hazelnut oil + coconut oil + stinging nettle oil:
Efficacy in the case of psoriasis

| Stinging nettle oil | | | | | |
|---|---|---|---|---|---|
| content in percent | 1 day | 5 days | 10 days | 20 days | 30 days |
| 1 | + | + | ++ | +++ | +++ |
| 2 | + | ++ | +++ | +++ | ++++ |
| 3 | ++ | +++ | ++++ | ++++ | ++++ |
| 5 | ++ | +++ | ++++ | ++++ | ++++ |
| 10 | ++ | +++ | +++ | ++++ | ++++ |

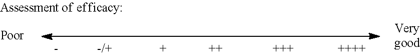

Overall, it is clearly apparent that the composition according to the invention demonstrates clearly improved results in the treatment of psoriasis compared with comparison compositions, after just 30 days. Similar results were also found when the compositions described here were used in the treatment of neurodermatitis or wounds/skin burns/corns.

The features of the invention which are disclosed in the above description and in the claims can be of significance both individually and in any combination for the realisation of the invention in its individual embodiments.

1. Composition to be applied to the skin, comprising a dermatologically compatible vehicle, coconut oil, hazelnut oil and/or avellana oil, and stinging nettle oil.

2. Composition according to claim 1, characterised in that it furthermore comprises TRF extract (tocotrienol-rich fraction).

3. Composition according to claim 1, characterised in that the percentage ratio of dermatologically compatible vehicle to coconut oil to hazelnut oil and/or avellana oil to stinging nettle oil lies within a range of 1-50:1-30:1-50:1-10.

4. Composition according to claim 1, characterised in that it furthermore comprises almond oil.

5. Composition according to claim 4, characterised in that the percentage ratio of vehicle to coconut oil to hazelnut oil and/or avellana oil to stinging nettle oil to almond oil lies within a range of 1-50:1-30:1-50:1-10:1-20.

6. Composition according to claim 1, characterised in that it furthermore comprises oil of bitter almonds.

7. Composition according to claim 6, characterised in that the weight ratio of vehicle to coconut oil to hazelnut oil and/or avellana oil to stinging nettle oil to almond oil to oil of bitter almonds lies within a range of 1-50:1-30:1-50:1-10:0-20:1-10.

8. Composition according to claim 1, characterised in that it furthermore comprises natural aromatics, preferably lavender aroma.

9. Composition according to claim 1, which comprises:
Dermatologically compatible vehicle 1-50% by weight,
Coconut oil 1-30% by weight,
Hazelnut oil and/or avellana oil 1-50% by weight,
Stinging nettle oil 1-10% by weight,
Almond oil 0-20% by weight,
TRF extract (tocotrienol-rich fraction) 0-20% by weight,
Oil of bitter almonds 0-10% by weight,
Aromatic substance 0-1% by weight,
wherein all the details of percentages by weight relate to the total quantity of the composition.

10. Composition according to claim 1, characterised in that it is presented in the form of an ointment, cream, lotion, tincture, oil or gel.

11. Composition according to claim 1, characterised in that the dermatologically compatible vehicle is selected from the groups of:
a. Hydrophobic ointments,
b. Lipophilic gels,
c. lipogels,
d. water-absorbing ointments W/O absorption ointments,
e. O/W absorption ointments,
f. Lipophilic creams,
g. W/O lotions,
h. quasi-W/O creams,
i. hydrophilic creams,
j. hydrophilic lotions, and
k. hydrophilic gels.

12. Composition according to claim 1 for use as a medication for the treatment of skin diseases, in particular psoriasis, neurodermatitis, atopic dermatitis, seborrhoeic dermatitis, urticaria, erythema and lichen planus, as well as for the treatment of wounds/skin burns and corns.

13. Use of the composition according to claim 1 for the treatment of skin diseases, in particular psoriasis, neurodermatitis, atopic dermatitis, seborrhoeic dermatitis, urticaria, erythema and lichen planus, as well as for the treatment of wounds/skin burns and corns.

14. Composition according to claim 9, which comprises:
Dermatologically compatible vehicle preferably 30-50% by weight,
Coconut oil preferably 10-30% by weight,
Hazelnut oil and/or avellana oil preferably 10-40% by weight,
Stinging nettle oil preferably 1-7% by weight
Almond oil preferably 5-15% by weight,
TRF extract (tocotrienol-rich fraction) preferably 1-10% by weight
Oil of bitter almonds preferably 0.5-3% by weight,
Aromatic substance preferably 0.3-0.7% by weight,
wherein all the details of percentages by weight relate to the total quantity of the composition.

15. Composition according to claim 9, which comprises:
Dermatologically compatible vehicle even more preferably 40.00% by weight,
Coconut oil even more preferably 17.50% by weight,
Hazelnut oil and/or avellana oil even more preferably 20-30% by weight,
Stinging nettle oil even more preferably 3.00% by weight,
Almond oil even more preferably 10.00% by weight,
TRF extract (tocotrienol-rich fraction) even more preferably 2-7% by weight,
Oil of bitter almonds even more preferably 1.00% by weight,
Aromatic substance even more preferably 0.50% by weight,
wherein all the details of percentages by weight relate to the total quantity of the composition.
